(19) United States
(12) Patent Application Publication
(10) Pub. No.: US 2005/0165472 A1
(43) Pub. Date: Jul. 28, 2005

(54) Radiopaque coating for biomedical devices

(76) Inventor: David A. Glocker, W. Henrietta, NY (US)

Correspondence Address: Mauri A. Sankus;JAECKLE FLEISCHMANN & MUGEL, LLP, 190 Linden Oaks, Rochester, NY, 14625-2812, US

(21) Appl. No.: 11/040,433

(22) Filed: Jan. 21, 2005

Related U.S. Application Data
(60) Provisional application No. 60/538,749, filed on Jan. 22, 2004.

(51) Int. Cl. A61F 2/06

(52) U.S. Cl. 623/1.15

(57) ABSTRACT

A medical device has a radiopaque coating that can withstand the high strains inherent in the use of such devices without delamination. A coating of Ta is applied to a medical device, such as a stent, by vapor deposition so that the thermomechanical properties of the stent are not adversely affected.

### CROSS REFERENCE TO RELATED APPLICATIONS

This application claims the benefit of U.S. Provisional Application No. 60/538,749.

### TECHNICAL FIELD

The present invention relates to medical devices.

### BACKGROUND

Stents have become extremely important devices in the treatment of cardiovascular disease. A stent is a small mesh “scaffold” that can be positioned in an artery to hold it open, thereby maintaining adequate blood flow. Typically a stent is introduced into the patient's system through the brachial or femoral arteries and moved into position using a guidewire. This minimally invasive procedure replaces surgery and is now used widely because of the significant advantages it offers for patient care and cost.

In order to deploy a stent, it must be collapsed to a fraction of its normal diameter so that it can be manipulated into the desired location. Therefore, many stents and guidewires are made of an alloy of nickel and titanium, known as nitinol, which has the unusual properties of superelasticity and shape memory. Both of these properties result from the fact that nitinol exists in a martensitic phase below a first transition temperature, known as M_{f}, and an austenitic phase above a second transition temperature, known as A_{f}. Both M_{f} and A_{f} can be manipulated through the ratio of nickel to titanium in the alloy. In the martensitic phase nitinol is very ductile and easily deformed, while in the austenitic phase it has a high elastic modulus. Applied stresses produce some martensitic material at temperatures above A_{f} and when the stresses are removed the material returns to its original shape. This results in a very springy behavior for nitinol, referred to as superelasticity. Furthermore, if the temperature is lowered below M_{f} and the nitinol is deformed, when the temperature is raised above A_{f} it will recover its original shape. This is described as shape memory. Stents having superelasticity and shape memory can be compressed to small diameters, moved into position, and deployed so that they recover their full size. By choosing an alloy composition having an A_{f} below normal body temperature, the stent will remain expanded with significant force once in place. Remarkably, during this procedure the nitinol must typically withstand strain deformations of as much as 8%.

FIG. 1 illustrates one of many stent designs that are used to facilitate this compression and expansion. This design uses ring shaped “struts,” 10 each one having corrugations that allow it to be collapsed to a small diameter. Bridges, a.k.a. nodes, 20 which also must flex in use, connect the struts 10. Many other types of expandable geometries are known in the field and are used for various purposes.

One disadvantage of stents made from nitinol is that both nickel and titanium have low atomic numbers and are, therefore, relatively poor X-ray absorbers. Consequently, nitinol stents of typical dimensions are difficult or impossible to see with X-rays when they are being manipulated or are in place. There are many advantages that would result from being able to see a stent in an X-ray image. For example, radiopacity, as it is called, would result in the ability to precisely position the stent initially and in being able to identify changes in shape once it is in place that may reflect important medical conditions.

Many methods are described in the prior art for rendering stents or portions of stents radiopaque. These include filling cavities on the stent with radiopaque material (U.S. Pat. No. 6,635,082; U.S. Pat. No. 6,641,607), radiopaque markers attached to the stent (U.S. Pat. No. 6,293,966; U.S. Pat. No. 6,312,456; U.S. Pat. No. 6,334,871; U.S. Pat. No. 6,361,557; U.S. Pat. No. 6,402,777; U.S. Pat. No. 6,497,671; U.S. Pat. No. 6,503,271; U.S. Pat. No. 6,554,854), stents comprised of multiple layers of materials with different radiopacities (U.S. Pat. No. 6,638,301; U.S. Pat. No. 6,620,192), stents that incorporate radiopaque structural elements (U.S. Pat. No. 6,464,723; U.S. Pat. No. 6,471,721; U.S. Pat. No. 6,540,774; U.S. Pat. No. 6,585,757; U.S. Pat. No. 6,652,579), coatings of radiopaque particles in binders (U.S. Pat. No. 6,355,058), and methods for spray coating radiopaque material on stents (U.S. Pat. No. 6,616,765). All of the prior art methods for imparting radiopacity to stents significantly increase the manufacturing cost and complexity and/or render only a small part of the stents radiopaque.

The most efficient method would be to apply a conformal coating of a fully dense radiopaque material to all surfaces of the stent. The coating would have to be thick enough to provide good X-ray contrast, biomedically compatible and corrosion resistant. More challenging, however, it would have to be able to withstand the extreme strains in use without cracking or flaking and would have to be ductile enough that the important thermomechanical properties of the stent are preserved.

Physical vapor deposition techniques, such as sputtering, thermal evaporation and cathodic arc deposition, can produce dense and conformal coatings of radiopaque materials like gold, platinum, tantalum, tungsten and others. Physical vapor deposition is widely used and reliable. However, coatings produced by these methods do not typically adhere well to substrates that undergo strains of up to 8%, as required in this application. This problem is recognized in U.S. Pat. No. 6,174,329, which describes the need for protective coatings over radiopaque coatings to prevent the radiopaque coatings from flaking off when the stent is being used.

Another important limitation of radiopaque coatings deposited by physical vapor deposition is the temperature sensitivity of nitinol. As mentioned, shape memory biomedical devices are made with values of A_{f} close to but somewhat below normal body temperature. If nitinol is raised to too high a temperature for too long its A_{f} value will rise and sustained temperatures above 300-400 C will adversely affect typical A_{f} values used in stents. Therefore, the time-temperature history of a stent during the coating operation is critical. In the prior art it is customary to directly control the temperature of a substrate in such a situation, particularly one with a very low thermal mass such as a stent. This is usually accomplished by placing the substrate in thermal contact with a large mass, or heat sink, whose temperature is controlled. Because of its shape and structure, controlling the temperature of a stent during coating would be a challenging task. Moreover, the portion of the stent in contact with the heat sink would receive no coating and the resulting radiographic image could be difficult to interpret.

Accordingly, there is a need in the art for biomedical devices having radiopaque coatings thick enough to provide good X-ray contrast, biomedically compatible, and corrosion resistant. Further, the coating needs to withstand the extreme strains in use without cracking or flaking and be sufficiently ductile so that the thermo-mechanical properties of the device are preserved.

### SUMMARY

The present invention is directed towards a medical device having a radiopaque outer coating that is able to withstand the strains produced in the use of the device without delamination.

A medical device in accordance with the present invention can include a body at least partially comprising a nickel and titanium alloy and a Ta coating on at least a portion of the body; wherein the Ta coating is sufficiently thick so that the device is radiopaque and the Ta coating is able to withstand the strains produced in the use of the device without delamination. The Ta coating can consist primarily of the bcc crystalline phase. The coating thickness is preferably between approximately 3 and 10 microns. The device can be a stent or a guidewire, for example.

A process for depositing a Ta layer on a medical device consisting of the steps of: maintaining a background pressure of inert gas in a sputter coating system containing a Ta sputter target; applying a voltage to the Ta target to cause sputtering; and sputtering for a period of time to produce the desired coating thickness. The device preferably is not directly heated or cooled and the equilibrium temperature of the device during deposition is controlled indirectly by the process. The equilibrium temperature preferably is between 150° and 450° C. A voltage, ac or dc, can be applied to the medical device during the process. An initial high voltage, preferably between 300 and 500 volts, can be applied to preclean the device for a first period of time, preferably between 1 minute and 20 minutes. A second, lower voltage, preferably between 50 and 200 volts, can be applied for a period of time, preferably between 1 and 3 hours. Preferably, the inert gas is from the group comprising Ar, Kr and Xe. Preferably, the voltage on the target(s) produces a deposition rate of 1 to 4 microns per hour. The target preferably is a cylinder or a plate.

A medical device comprises a body having an outer layer and a radiopaque coating on at least a portion of the outer layer; wherein the coating is applied using a physical vapor deposition technique.

### BRIEF DESCRIPTION OF THE DRAWINGS

These and other features, aspects and advantages of the present invention will become better understood with regard to the following description, appended claims, and accompanying drawings where:

FIG. 1 illustrates a stent found in the prior art;

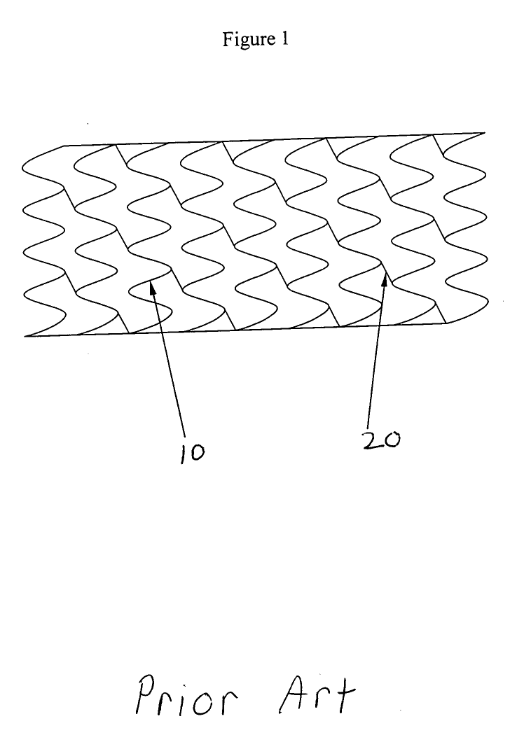

FIG. 2 illustrates a Ta target surrounding a stent; and

FIG. 3 illustrates a cross section of a conformal coating of Ta on a strut 10 of the stent in FIG. 1.

### DESCRIPTION

This patent relates to coatings that render biomedical devices radiopaque and that withstand the extremely high strains inherent in the use of such devices without delamination. Specifically, it relates to coatings of Ta having these properties and methods for applying them that do not adversely affect the thermomechanical properties of stents.

Tantalum has a high atomic number and is also biomedically inert and corrosion resistant, making it an attractive material for radiopaque coatings in this application. It is known that Ta coatings between 3 and 10 microns thick provide adequate radiopacity on stents. However, because Ta has a melting point of almost 3000 C, any coating process must take place at a low homologous temperature (the ratio of the deposition temperature to the melting temperature in degrees Kelvin) to preserve the A_{f} values of the stents as described previously. It is well known in the art of physical vapor deposition that low homologous coating temperatures often result in poor coating properties. Nevertheless, we have unexpectedly found that radiopaque Ta coatings deposited under the correct conditions are able to withstand the strains inherent in stent use without flaking.

Still more remarkable is the fact that we can deposit these adherent coatings at high rates with no direct control of the stent temperature without substantially affecting A_{f}. For a thermally isolated substrate, the equilibrium temperature will be determined by factors such as the heat of condensation of the coating material, the energy of the atoms impinging on the substrate, the coating rate, the radiative cooling to the surrounding chamber and the thermal mass of the substrate. It is surprising that this energy balance permits high-rate coating of a temperature sensitive low mass object such as a stent without raising the temperature beyond acceptable limits. Eliminating the need to directly control the temperature of the stents significantly simplifies the coating operation and is a particularly important consideration for a manufacturing process.

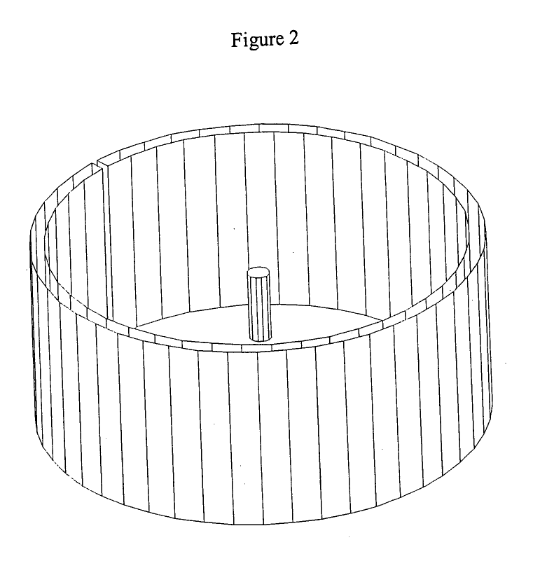

An inverted cylindrical magnetron sputtering system, as is well-known in the art, was used to deposit the coatings. An example of this type of system is described in Surface and Coatings Technology 146-147 (2001), pages 457-462. The cylindrical magnetron sputtering system used a single cylindrical magnetron driven with dc power to deposit the Ta. The cathode was 19 cm in diameter and 10 cm high. FIG. 2 illustrates the Ta target surrounding a stent as described herein. Other devices well known to those in the art, such as a vacuum chamber, vacuum pumps, power supplies, gas flow meters, pressure measuring equipment and the like, are omitted for clarity.

Prior to coating, the stents were cleaned with a warm aqueous cleaner in an ultrasonic bath and rinsed twice in ultrasonic water baths. The stents were blown dry with nitrogen and further dried with hot air.

Individual stents were held in the center of the coating chamber by a spring clip attached at one end. The system was evacuated to a base pressure no greater than 1.0×10⁻⁶ Torr. Either Kr or Xe was used as a sputtering gas at a pressure of 4.0 mTorr. The cylindrical magnetron cathode was operated at a power of 1.0 kW for the entire coating. A commercially pure (99.5%) Ta target was used.

The target was preconditioned at the process power and pressure for 10 minutes. During this step a shutter isolated the stents from the target. A_{f} ter the shutter was opened, the first few minutes of coating were applied using a bias voltage of −400 V applied to the stents. The remaining coating was applied with a bias voltage of −150 V applied to the stents. A coating time of 2 hours 15 minutes resulted in a coating thickness of approximately 10 microns. This is a very acceptable coating rate for a manufacturing process. The stents were not heated or cooled in any way during deposition and their time-temperature history was determined entirely by the coating process.

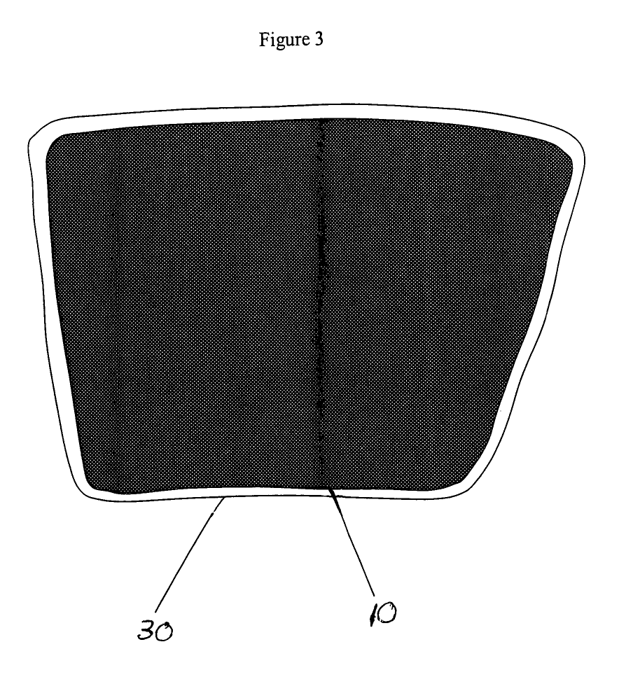

FIG. 3 illustrates the cross section of a conformal coating of Ta 30 on a strut 10, shown approximately to scale for a 10 micron thick coating. Stents coated in this manner were evaluated in several ways. First, they were pressed into adhesive tape and it was found that no coating was removed from the stent surfaces. We also saw that the stents came back to their original shape at room temperature after distortion, demonstrating that A_{f} was not affected significantly by the coating operation. Next, the stents were cooled in a dry ice/alcohol bath to a temperature of −46 C and stretched to their maximum length at this temperature. Because of their design, this flexed some of the struts in the same manner and to approximately the same degree that they would be flexed in use. The stents were then warmed to room temperature and examined under a microscope. No flaking or cracking was seen at the maximum flexure points. This procedure was repeated twice more with the same results.

While not wanting to be bound by this explanation, we believe that part of the reason for these surprising results is that these conditions produce a coating substantially made up of alpha Ta. Sputtered Ta typically exists in one of two crystalline phases, either tetragonal (known as the beta phase) or body centered cubic (bcc) (known as the alpha phase). The alpha phase of Ta is much more ductile than the beta phase and can therefore withstand greater strains. It is known in the art that sputtering Ta in Kr or Xe with substrate bias can result in the alpha phase being deposited at temperatures as low as 200 C. See, for example, Surface and Coatings Technology 146-147 (2001) pages 344-350. Even if this explanation is correct, there is nothing in the prior art or in our experience to suggest that alpha Ta coatings of 10 microns thickness can withstand the very high strains inherent in the use of stents without delamination and coating failure. There is also no indication in the prior art that a high-rate coating process such as this is possible on a delicate substrate such as a stent without raising the substrate temperature to an unacceptable level.

Although the present invention has been described in considerable detail with reference to certain preferred versions thereof, other versions are possible. For example, a device other than a stent can be coated with Ta or another radiopaque material. Therefore, the spirit and scope of the appended claims should not be limited to the description of the preferred versions contained herein.

All features disclosed in the specification, including the claims, abstracts, and drawings, and all the steps in any method or process disclosed, may be combined in any combination, except combinations where at least some of such features and/or steps are mutually exclusive.

Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” for “step” clause as specified in 35 U.S.C. § 112.

1) A medical device comprising:
a) a body at least partially comprising a nickel and titanium alloy; and
b) a Ta coating on at least a portion of the body; wherein the Ta coating is sufficiently thick so that the device is radiopaque and the Ta coating is able to withstand the strains produced in the use of the device without delamination.

2) Claim 1 in which said Ta coating consists primarily of the bcc crystalline phase.

3) Claim 1 in which said coating thickness is between approximately 3 and 10 microns.

4) Claim 1 in which said device is a stent.

5) Claim 1 in which said device is a guidewire.

6) A process for depositing a Ta layer on a medical device consisting of the steps of:
a) maintaining a background pressure of inert gas in a sputter coating system containing a Ta sputter target;
b) applying a voltage to said Ta target to cause sputtering; and
c) sputtering for a period of time to produce the desired coating thickness

7) Claim 6 in which said device is not directly heated or cooled and the equilibrium temperature of said device during deposition is controlled indirectly by said process.

8) Claim 7 in which said equilibrium temperature is between 150 and 450 C.

9) Claim 6 in which a voltage is applied to said medical device during said process.

10) Claim 9 in which said voltage comprises an initial high voltage to preclean said device for a first period of time.

11) Claim 10 in which said initial high voltage is between 300 and 500 volts

12) Claim 10 in which said first period of time is between 1 minute and 20 minutes.

13) Claim 9 in which said voltage comprises a second, lower voltage applied for a second period of time.

14) Claim 13 in which said lower voltage is between 50 and 200 volts

15) Claim 13 in which said second period of time is between 1 hour and 3 hours.

16) Claim 6 in which said inert gas is from the group comprising Ar, Kr and Xe

17) Claim 6 in which said voltage produces a deposition rate of 1 to 5 microns per hour

18) Claim 6 in which said voltage is dc

19) Claim 6 in which said voltage is ac.

21) Claim 6 in which said voltage is applied in pulses

22) Claim 6 in which said target is a cylinder.

23) Claim 6 in which said target is a plate.

24) A medical device comprising:
a) a body having an outer layer; and
b) a radiopaque coating on at least a portion of the outer layer; wherein the coating is applied using a physical vapor deposition technique.
